(19) United States
(12) Patent Application Publication
(10) Pub. No.: US 2017/0193578 A1
(43) Pub. Date: Jul. 6, 2017

(54) System and Distribution of Nutritional Supplements

(71) Applicant: ICON Health & Fitness, Inc., Logan, UT (US)

(72) Inventor: Scott R. Watterson, Logan, UT (US)

(21) Appl. No.: 15/392,486

(22) Filed: Dec. 28, 2016

Related U.S. Application Data
(60) Provisional application No. 62/273,852, filed on Dec. 31, 2015.

(51) Int. Cl. G06Q 30/06; A61B 5/0488; A61B 5/00; A61B 5/021; G09B 19/00; A63B 24/00

(52) U.S. Cl. CPC G06Q 30/0621; G09B 19/0092; A63B 24/0062; A61B 5/4869; A61B 5/021; A61B 5/0488; A63B 2230/06; A63B 2230/50; A63B 2230/42

(57) ABSTRACT

A system for customizing nutritional supplement includes a processor and memory. The memory includes programmed instructions that, when executed, cause the processor to access a profile of a user in a web based program; identify, from the profile, at least one physiological parameter experienced by the user within a recent time period; identify, from the profile, at least a portion of a workout history from the recent time period; identify a fitness goal of the user; and generate a customized nutritional supplement formula based on the physiological parameter, the portion of a workout history, and the fitness goal.

### RELATED APPLICATIONS

This application claims priority to U.S. Provisional Patent No. 62/273,852 filed on 31 Dec. 2015 and titled “System and Distribution of Nutritional Supplements.” U.S. Provisional Patent No. 62/273,852 is incorporated herein by reference for all that it discloses.

### BACKGROUND

While numerous exercise activities exist that one may participate in, exercise may be broadly broken into the categories of aerobic exercise and anaerobic exercise. Aerobic exercise generally refers to activities that substantially increase the heart rate and respiration of the exerciser for an extended period of time. This exercise is generally directed to enhancing cardiovascular performance. This exercise usually includes low or moderate resistance to the movement of the individual. For example, aerobic exercise includes activities such as walking, running, jogging, swimming or bicycling for extended distances and extended periods of time.

Anaerobic exercise generally refers to exercise that strengthens skeletal muscles and usually involves the flexing or contraction of targeted muscles through significant exertion during a relatively short period of time and/or through a relatively small number of repetitions. For example, anaerobic exercise includes activities such as weight training, push-ups, sit-ups, pull-ups, or a series of short sprints.

During aerobic and anaerobic exercise, the human body uses vitamins, minerals, water, carbohydrates, and other nutrients to cause the muscles to contract. Further, muscles are often damaged during exercise, and the body uses other types of nutrients, such as proteins, to repair muscles and/or otherwise recover from the workout. Thus, those who engage in exercise often try to consume nutritious foods to stay healthy.

One type of system for assisting with proper nutrition is disclosed in U.S. Pat. No. 5,954,640 issued to Andrew Szabo. Szabo describes a method for proposing and providing nutritional supplementation for a person including the steps of receiving personal information, e.g., relating to health and diet, about the person, determining a health model for the person, determining an effect on the health model for at least two nutritional supplements, optimizing a proposed nutritional supplementation for the person based on the personal information about the person and effect for at least two nutritional supplements, through employment of the health model, and outputting a proposed nutritional supplementation including amounts of at least two nutritional supplements. The method may also receive economic considerations, e.g., a budget, for the nutritional supplementation, and further optimize the nutritional supplementation based on the economic considerations. Other types of systems are described in U.S. Patent Publications No. 2011/0014351 issued to Carroll Reider, et al and 2014/0236759 issued to Christopher Mirabile.

### SUMMARY

In one embodiment, a system for customizing nutritional supplement includes a processor and memory. The memory includes programmed instructions that, when executed by the processor, cause the processor to access a profile of a user in a web based program; identify, from the profile, at least one physiological parameter experienced by the user within a recent time period; identify, from the profile, at least a portion of a workout history from the recent time period; identify a fitness goal of the user; and generate a customized nutritional supplement formula based on the physiological parameter, the portion of a workout history, and the fitness goal.

The programmed instructions may cause the processor, when executed, to determine consumed nutrient amounts by the user based on at least one physiological parameter and workout history, determine goal nutrient amounts to achieve the fitness goal, and combine the consumed nutrient amounts and the goal nutrient amounts.

Identifying the fitness goal may include extracting goal information from the profile.

Identifying the fitness goal may include receiving the fitness goal from a source other than the profile.

Identifying at least one physiological parameter may include identifying an amount of sleep of the user within the recent time period.

Identifying at least one physiological parameter may include identifying a heart rate of the user during an event in the workout history.

Identifying at least one physiological parameter may include identifying a user temperature during sleep within the recent time period.

Identifying at least one physiological parameter may include identifying a user temperature during an event in the workout history.

Identifying at least one physiological parameter may include identifying a user respiration rate during an event in the workout history.

Identifying at least one physiological parameter may include identifying a user weight within the recent time period.

Identifying at least one physiological parameter may include identifying a user gender.

Identifying at least one physiological parameter may include identifying a user body composition within the recent time period.

The programmed instructions may further track a dietary input of the user, identify any dietary nutritional deficiencies of the user, and add a nutrient to the customized nutritional supplement formula in response to the identified dietary nutritional deficiency.

The system may further include multiple containers, wherein each of the multiple containers is filled with at least one type of nutrient and a closable orifice in at least one of the multiple containers. The memory includes programmed instructions that cause the processor, when executed, to direct at least one type of nutrient into a supplement combination based on the customized nutritional supplement formula.

The memory may include programmed instructions that, when executed, cause the processor to prepare a supplement combination based on the customized nutritional supplement formula to be shipped to the user.

Preparing the supplement combination may include printing a shipping address associated with the user.

In one embodiment, a system for customizing nutritional supplement includes a processor and memory. The memory includes programmed instructions that, when executed, cause the processor to access a profile of a user in a web based program; identify, from the profile, at least one physiological parameter experienced by the user within a recent time period; identify, from the profile, at least a portion of a workout history from the recent time period; determine consumed nutrient amounts by the user based on at least one physiological parameter and workout history; identify a fitness goal of the user; determine goal nutrient amounts to achieve the fitness goal; and combine the consumed nutrient amounts and the goal nutrient amounts to generate a customized nutritional supplement formula.

The system may further include multiple containers, wherein each of the multiple containers is filled with at least one type of nutrient and a closable orifice in at least one of the multiple containers. The memory may include programmed instructions that cause the processor, when executed, to direct at least one type of nutrient into a supplement combination based on the customized nutritional supplement formula.

The memory may include programmed instructions that, when executed, cause the processor to prepare a supplement combination based on the customized nutritional supplement formula to be shipped to the user.

Preparing the supplement combination may include printing a shipping address associated with the user.

In one embodiment, a system for customizing nutritional supplement includes multiple containers, wherein each of the multiple containers is filled with at least one type of nutrient, a closable orifice in at least one of the multiple containers, and a processor and memory. The memory includes programmed instructions that, when executed, cause the processor to access a profile of a user in a web based program; identify, from the profile, at least one physiological parameter experienced by the user within a recent time period; identify, from the profile, at least a portion of a workout history from the recent time period; determine consumed nutrient amounts by the user based on at least one physiological parameter and workout history; identify a fitness goal of the user; determine goal nutrient amounts to achieve the fitness goal; combine the consumed nutrient amounts and the goal nutrient amounts to generate a customized nutritional supplement formula; direct at least one type of nutrient into a supplement combination based on the customized nutritional supplement formula; and prepare a supplement combination to be shipped to the user by printing a shipping address associated with the user.

In one embodiment, a method for customizing a nutritional supplement to a user includes identifying at least one physiological parameter experienced by the user within a recent time period from a web based program; identifying at least a portion of a workout history from the recent time period from the web based program; identifying a fitness goal of the user from the web based program; combining nutritional supplements to form a nutritional supplement combination based on the at least one physiological parameter, the portion of the workout history, and the fitness goal; and shipping the nutritional supplement combination to the user.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates a diagram of an example of a customizing system in accordance with the present disclosure.

FIG. 2 illustrates a diagram of an example of a personal profile in accordance with the present disclosure.

FIG. 3 illustrates a diagram of an example of a nutritional combination in accordance with the present disclosure.

FIG. 4 illustrates a diagram of an example of a dispensing system in accordance with the present disclosure.

FIG. 5 illustrates a diagram of an example of a method for customizing a nutritional combination in accordance with the present disclosure.

FIG. 6 illustrates a diagram of an example of a customizing system in accordance with the present disclosure.

FIG. 7 illustrates a diagram of an example of a method for customizing a nutritional combination in accordance with the present disclosure.

Throughout the drawings, identical reference numbers designate similar, but not necessarily identical, elements.

### DETAILED DESCRIPTION

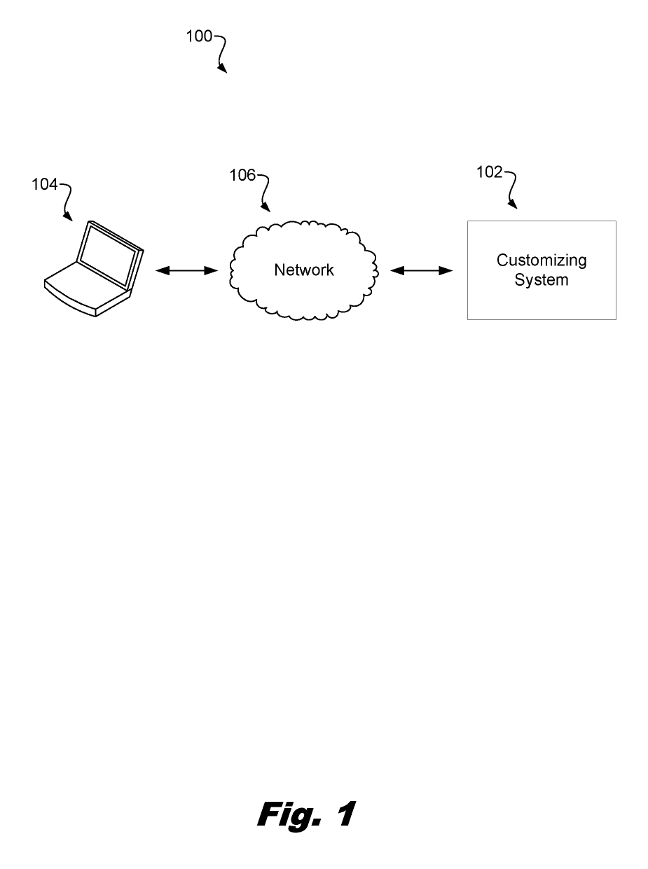

FIG. 1 depicts an example of an environment 100 in which the present systems and methods may be implemented. In some embodiments, the environment 100 includes a customizing system 102 in communication with a remote computer 104 over a network 106. Any appropriate mechanism for communicating between the remote computer 104 and the customizing system 102 may be used. In example of FIG. 1, a wireless network is used to communicate between the remote computer 104 and the customizing system 102. The remote computer 104 may include a personal profile of a user, and the customizing system 102 may extract data from the profile information about the user from the remote computer 104.

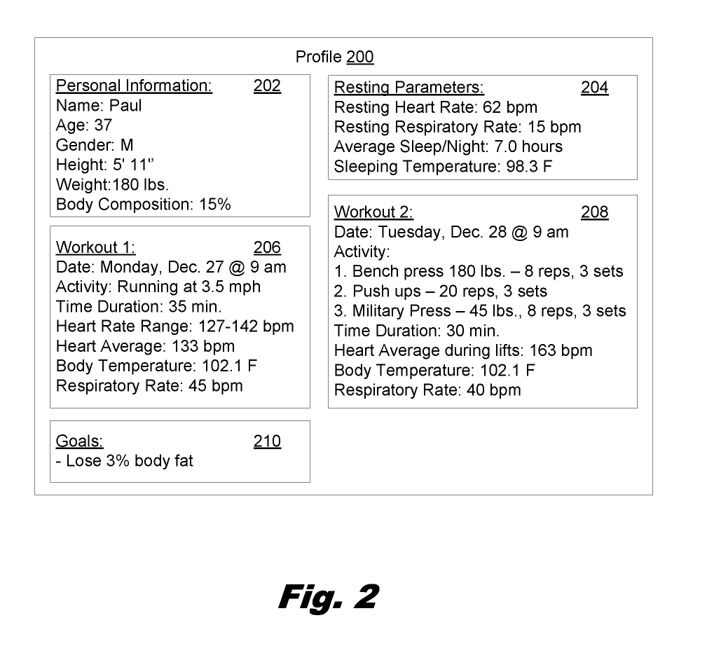

FIG. 2 depicts an example of a personal profile 200 of a user. In this example, the personal profile includes personal information 202. In this example, the personal information includes the user's name, age, gender, height, weight, and body composition. Also, a user's resting parameters 204 are also depicted in the illustrated example. The resting parameters include a resting heart rate, a resting respiratory rate, an average amount of sleep per night, and a sleeping temperature. The personal profile 200 may also include information about the user's workout history. In this example, the workout history includes a first workout 206 and a second workout 208. The profile 200 also includes a user goal 210 of losing three percent body fat.

The first workout 206 is depicted as occurring on Monday, December 27th at 9:00 am. The first workout 206 included an activity of running at 3.5 miles per hour for 35 minutes. During the user's first workout 206, the user's heart rate ranged between 127 to 142 beats per minute with the user's average heart rate being at 133 beats per minute. During the first workout 206, the user's body temperature rose to 102 degrees Fahrenheit, and a respiratory rate of 45 breaths per minute.

The second workout 208 is depicted as occurring on Tuesday, December 28th at 9:00 am. The activity includes bench pressing 180 pounds at eight repetitions for three sets, doing three sets of 20 push-ups for each set, and doing the military press with 45 pounds for eight repetitions at three sets. The second workout 208 lasted for 30 minutes. During the second workout, the user's average heart rate during the lifts was 163 beats per minute, and the user's body temperature was 102.1 degrees Fahrenheit. The user's respiratory rate was 25 breaths per minute during the lifts.

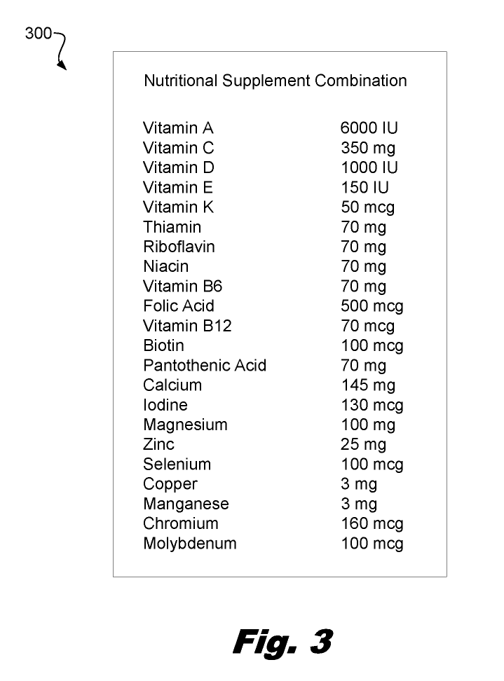

FIG. 3 depicts a nutritional supplement combination 300 that is based on at least one of the user's physiological parameters and the user's workout history. In this example, the customized nutritional supplement combination includes 6,000 international units of vitamin A, 350 milligrams of vitamin C, 1,000 international units of vitamin D, 150 international units vitamin E, 50 micrograms of vitamin K, 70 milligrams of thiamin, 70 milligrams of riboflavin, 70 milligrams of niacin, 70 milligrams of vitamin B6, 500 micrograms of folic acid, 70 milligrams of vitamin B12, 100 micrograms of biotin, 70 milligrams of pantothenic acid, 145 milligrams of calcium, 130 micrograms of iodine, 100 milligrams of magnesium, 25 milligrams of zinc, 100 micrograms of selenium, 3 milligrams of copper, 3 milligrams of manganese, 160 micrograms of chromium, and 100 micrograms of molybdenum.

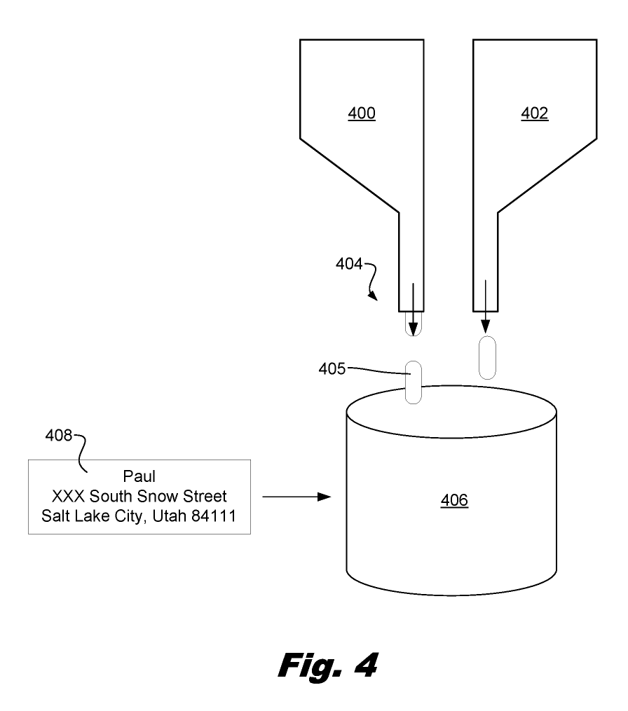

FIG. 4 depicts an example of a first container 400 of a first type of nutritional supplement and a second container 402 of a second type of nutritional supplement. In other examples, the system may include more than just a first and second container with additional types of nutritional supplements. In response to the system determining a nutritional supplement formula, the system can trigger at least one of the containers to open a closable orifice 404 to direct the nutritional supplement within the container to become part of a nutritional supplement combination that is based on the formula. For example, in those situations where the formula calls for a combination that can be completed in part with the supplements in the first container, the closable orifice may open long enough to release an appropriate amount of the type of supplement in the first container. Likewise, in situations where the nutritional supplement combination can be completed, at least in part, with the nutritional supplement stored in the second container, the closable opening of the second container can be opened just long enough to release the type of nutritional supplement within the second container. In the example of FIG. 4, the supplements in the containers are stored in capsules 405. In other examples, the containers may hold the nutritional supplements in merely a powdered form, a liquid form, or in another type of form. Further, the containers may include a single type of nutrient, like a single type of mineral or a single type of vitamin. In other examples, the nutrients in each container and/or each capsule may include a blend of different types of nutrients.

The released nutritional supplements can be directed to packaging 406 intended to be shipped to the user. Thus, the directed nutritional supplements may be prepared to be sent to the user by packaging the nutritional supplement combination. A shipping label 408 may be attached to the packaging 406 to prepare the packaging 406 for shipping.

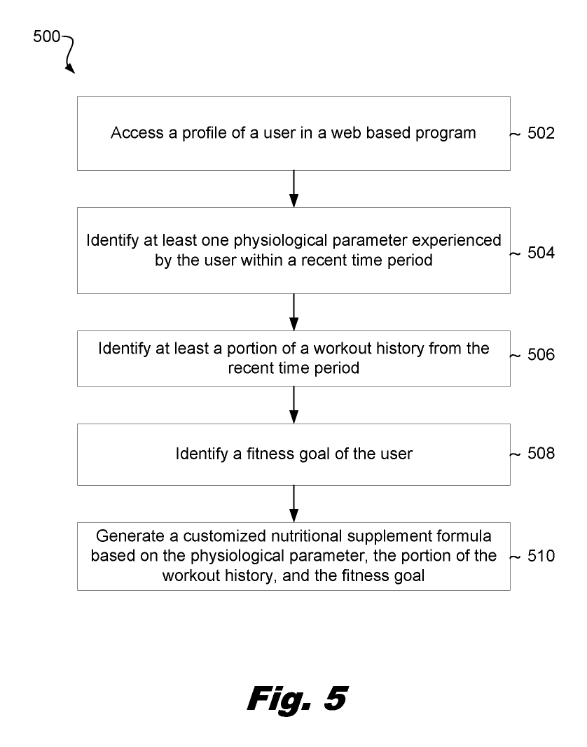

FIG. 5 depicts an example of a method 500 for customizing a nutritional supplement formula. In this example, the method 500 includes, accessing 502 a profile of a user in a web based program, identifying 504 at least one physiological parameter experienced by the user within a recent time period, identifying 506 at least a portion of a workout history from the recent time period, identifying 508 a fitness goal of the user, and generating 510 a customized nutritional supplement formula based on the physiological parameter, the portion of the workout history, and the fitness goal.

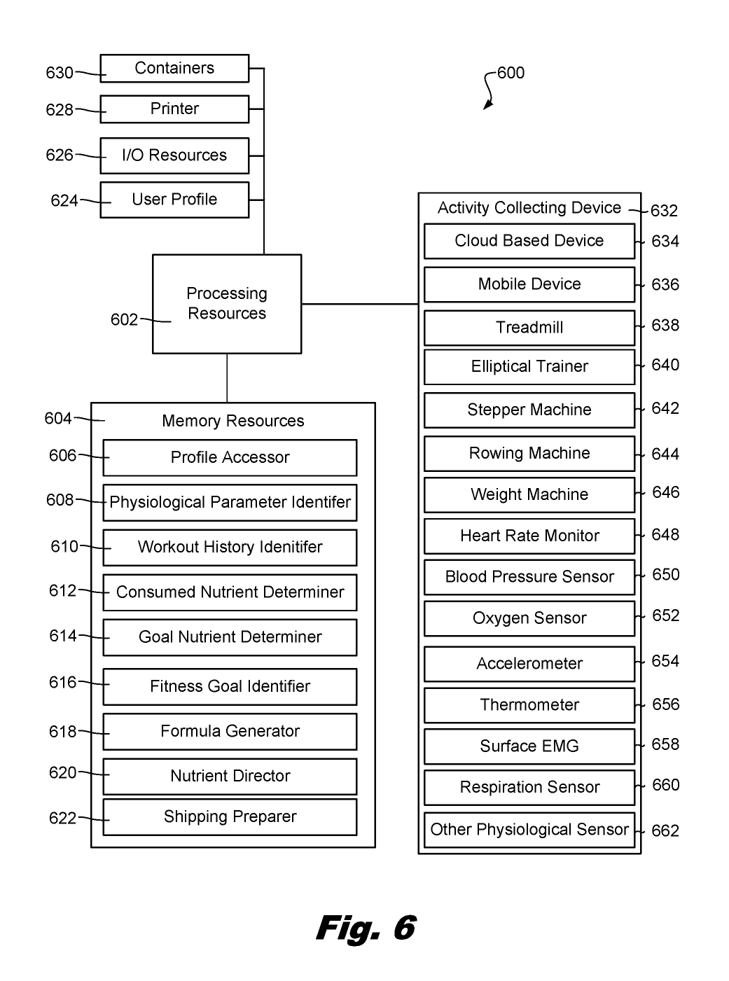

FIG. 6 depicts an example of a customizing system 600. In this example, the customizing system 600 includes processing resources 602 and memory resources 604. The memory resources 604 may cause the processing resources 602 to carry out functions programmed in the memory resources 604. In this example, the memory resources 604 include a profile accessor 606, a physiological parameter identifier 608, a workout history identifier 610, a consumed nutrient determiner 612, a goal nutrient determiner 614, a fitness goal identifier 616, a formula generator 618, a nutrient director 620, and a shipping preparer 622.

The processing resources 602 may be in communication with I/O resources 626, which may include a receiver, a transmitter, a transceiver, another type of communication device, or combinations thereof. Further, the processing resources 602 may be in direct communication or in communication through the I/O resources with container 630, a printer 628, and a user profile 624. The processing resources 602 may also be in communication with an activity collecting device 632. The activity collecting device 632 may include a cloud based device 634, a mobile device 636, a treadmill 638, an elliptical trainer 640, a stepper machine 642, a rowing machine 644, a weight machine 646, a heart rate monitor 648, a blood pressure sensor 650, an oxygen sensor 652, an accelerometer 654, a thermometer 656, a surface electromyography sensor 658, a respiration sensor 660, and other types of physiological sensors 662.

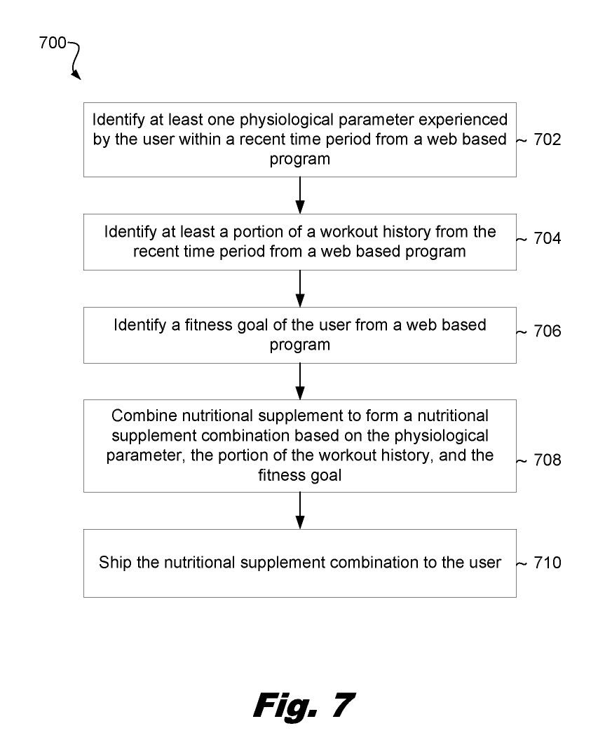

In the example of FIG. 7, a method 700 for customizing a nutritional supplement to a user includes identifying 702 at least one physiological parameter experienced by the user within a recent time period from a web based program; identifying 704 at least a portion of a workout history from the recent time period from the web based program; identifying 706 a fitness goal of the user from the web based program; combining 708 nutritional supplements to form a nutritional supplement combination based on the at least one physiological parameter, the portion of the workout history, and the fitness goal; and shipping 710 the nutritional supplement combination to the user.

### General Description

In general, the invention disclosed herein may provide users with a system that calculates a personalized nutritional supplement formula based on at least one parameter of the user's physiological history, the user's workout history, and the user's goals. The system generates a nutritional supplement combination based on the nutritional supplement formula, and sends the nutritional supplement combination to the user. The user's physiological information and workout history information can be identified by accessing the user's personal profile. The personal profile may be part of a fitness tracking program, a social media program, another type of program, parts of multiple programs, or combinations thereof.

After a fitness activity, a user is often depleted of nutrients. Generally, the degree of depletion depends on the intensity and type of fitness activity performed. Often, the user desires to replenish the nutrients lost during a workout, but may not know how many nutrients were actually lost.

The principles described in the present disclosure include a system that determines the amount and types of nutrients lost by a user during the user's workout. For example, if the user often completes endurance workouts, the user may lose a combination of carbohydrates and proteins as well as specific minerals and vitamins during each of the endurance workouts. In situations where an activity collecting device tracks parameters of the workout, the information about the workout can be analyzed. Over a period of time, the system can determine the types of workouts generally performed by the user. Thus, the system can customize a nutritional supplement combination that is well suited for the types of workouts performed by the user.

In some examples, the system tracks more than just the fitness activities performed by the user during a workout. For example, the system may also track aspect of other types of activities performed by the user. For example, the system may collect information about the user during rest, such as the user's resting heart rate, the user's resting temperature, the user's temperature during sleep, the user's average amount of sleep, the user resting breaths per minute, other physiological parameters about the user during rest, or combinations thereof. This information may be collected with activity trackers, inputs into a website, collected through another mechanism, or combinations thereof.

In one embodiment, the collected data is stored in a personal profile that may be in communication with activity trackers that collect the information. The activity trackers may include smart watches, sensors, mobile devices, physiological sensors incorporated into exercise machines (e.g. treadmills, elliptical trainers, stepper machines, rowing machines, etc.), or combinations thereof. The personal profile may also include information about the user's age, weight, height, gender, body composition, health conditions, hereditary information, other types of personal information, or combinations thereof. Further, an activity collecting device may gather data about the user from different sources.

In one example, the activity collecting device may include a component that tracks the physical activity performed by the user in real time and additionally stores activities that a user has already done. Further, the activity collecting device may also include a component that can receive fitness goals, workout schedules, planned workout activities, health conditions, or combinations thereof. The additional information may be inputted into the activity collecting device through a user interface, which may be located locally on the activity collecting device or remotely, such as through a wireless connection.

In some examples, a fitness activity is a workout, which can include anaerobic exercises, aerobic exercises, or combinations thereof. Further, the fitness activity may include playing basketball, dodge ball, baseball, football, capture the flag, volleyball, badminton, tennis, softball, soccer, hockey, water polo, other activities, or combinations thereof. Further, the fitness activity may include other active activities often performed for recreational purposes such as wrestling, fencing, curling, skiing, running, walking, swimming, stretching, rowing, dancing, golfing, horseback riding, kayaking, skateboarding, snorkeling, diving, staking, scuba diving, rock climbing, boxing, snow shoeing, martial arts, yoga, other activities, or combinations thereof. For the purposes of this disclosure, other types of activities may also be recorded as such daily activities such as gardening, shoveling, watching television, sleeping, standing, reading, eating, playing with kids, cleaning, cooking, shopping, performing home repairs, other types of activities, or combinations thereof. While some of these activities are not physically intense, an activity collecting device can nevertheless factor in an exertion by the user in performing these activities to determine the amount of nutrients for the user.

After a predetermined time period is past, the information about the user's activity and physiology can be analyzed to determine the amount of nutrients used during the the predetermined amount of time. Based in part on these determined amounts and types of nutrients depleted by the user, the system can generate a nutritional supplement formula. This information may be gathered by the customizing system that accesses the user's personal profile. In some cases, the customizing system also has access to the user's personal goals. In those circumstances where the customizing system has access to the user's personal goals, the customizing system may use the goals as a factor for determining the nutritional supplement formula.

The user's goals may influence the amount or types of nutrients included in the nutritional supplement formula. For example, when the user has a muscle gain goal, the nutritional supplement formula may include more protein, amino acids, and other nutrients understood to contribute to building muscle faster. In another example where the user's goal is to lose fat, the nutritional supplement formula may include supplements with a reduced amount of sugar, appetite suppressants, and/or other types of nutrients understood to enhance the user's ability to lose fat.

Additionally, at least one of the user's physiological parameters can also be used to determine the nutritional supplement formula. For example, the user's weight and/or body composition may be used to determine the amount of vitamins and minerals that the user can absorb after a workout. Additionally, a health condition may influence the nutritional supplement formula to adjust certain types of nutrients amounts to treat that health condition. For example, if the user has anemia, the nutritional supplement formula may include an increased amount of zinc and/or iron. Also, in those situations where the user is getting less sleep than is ideal, unrestful sleep, or has a temperature during sleep that is outside of typical temperatures for sleep, the nutritional supplement formula can include nutrients to improve the user's ability to sleep at night. Nutrients that may improve the user's sleep may include magnesium, inositol, B vitamins, thiamine, valerian, chamomile, ginseng, l-theanine, other nutrients, or combinations thereof. Another physiological parameter that may influence the user's nutritional supplement formula includes the user's resting temperature and/or the user's temperature during a workout. Nutrients that may improve temperature related issues may include coenzyme B vitamins, malic acid, magnesium, iron, zinc, selenium, l-tyrosine, manganese, iodine, other nutrients, or combinations thereof.

The customizing system may determine when to initiate the process of determining the nutritional supplement formula at any appropriate time. In some cases, the user makes a request for the system to determine the nutrient supplement formula. In other examples, times for determining the formula are predetermined at specific time periods. In yet other examples, the system determines when to generate the formula based on when the user appears to be running low on his or her nutritional supplements.

In response to determining the nutritional supplement formula, the system may initiate a protocol causing the nutritional supplement combination to be packaged and shipped to the user. In some situations, the nutritional supplement combinations is mixed from various types of separate nutritional supplements. In other examples, the system may select an appropriate supplement combination from a number of pre-mixed supplement combinations that fit the user's needs based on the user's physiological parameters, workout history, and the user's goals.

The system may include multiple containers that include multiple types of nutritional supplements. In response to determining the nutritional supplement formula, the system may cause a closable orifice in at least one of the multiple containers to open to direct the nutrients within the container towards packaging to send to the user. In some cases, multiple containers may be opened to retrieve multiple types of nutrients. While this example has been described with reference to opening a closable orifice to release the nutrients, the nutrients may be retrieved from the containers through any appropriate mechanism. For example, the nutrients may be removed with an auger, a conveyor, a chute, another type of retrieval mechanism, or combinations thereof.

In those examples where the nutrients are removed from the containers with a closable orifice, the orifice can be opened by activating an actuator in communication with a processor. In some examples, the actuator may be a solenoid, a screw motor, a linear actuator, a magnetic actuator, a hydraulic actuator, a pneumatic actuator, another type of actuator, or combinations thereof.

While the above examples describe the nutrients being in the form of a powder or capsule, the nutrient may take any appropriate form. For example, the nutrients may be dispensed in the form of a pill, paste, sauce, slurry, pellet, liquid, mist, other form, or combinations thereof. Further, the nutrients can be dispensed into a shipping container or into another type of container.

The shipping container may include a set of instructions with recommendations for when the user is to take the nutritional supplements. Additionally, the shipping container may include information about the supplement combination, such as the types and amounts of nutrients in the supplement combination. In some cases, the information may include a restatement of the user's goals so that the user can ensure that the supplements are customized to his or her goals. Likewise, the instructions may also include a statement about the user's physiological parameter and/or workout history to also allow the user to confirm that the supplement combination matches the information that the user has.

The system may also cause a printer to print a label that includes the user's shipping address. This label may be applied to the packaging, so that the packaging containing the nutritional supplement combination can be sent to the user.

The nutrients in the nutritional supplement combination may include any appropriate type of nutrients. For example, the nutrients may include creatine kinase, protein, water, carbohydrates, sodium, chloride, potassium, magnesium, calcium, phosphate, vitamin A, vitamin, B, vitamin C, vitamin D, vitamin E, vitamin K, thiamin, riboflavin, niacin, folate, pyridoxine, cobalamin, pantothenic acid, biotin, iron, zinc, copper, selenium, iodine, fluoride, chromium, manganese, molybdenum, magnesium, glutamine, glycine, carnitine, arginine, taurine, lysine, calcium, lutein, other vitamins, other minerals, other amino acids, other proteins, other herbs, other hormones, other molecules, other carbohydrates, other nutrients, or combinations thereof.

In some examples, a system for customizing the nutritional supplement formula includes accessing a profile of a user in a web based program, identifying at least one physiological parameter experienced by the user within a recent time period, identifying at least a portion of the workout history from the recent time period, identifying a fitness goal of the user, and generating a customized nutritional supplement formula based on the physiological parameter, the portion of the workout history, and the fitness goal.

In some examples, the web based program is a fitness tracking program. An example of a program that may be compatible with the principles described herein includes the iFit program which is available through www.ifit.com. An example of a program that may be compatible with the principles described in this disclosure is described in U.S. Pat. No. 7,980,996 issued to Paul Hickman. U.S. Pat. No. 7,980,996 is herein incorporated by reference for all that it discloses. In some examples, the user information accessible through the remote device includes the user's age, gender, body composition, height, weight, health conditions, other types of information, or combinations thereof. In some examples, the user's goals are also included in the profile. But, in alternative examples, the user goals are obtained through another source. For example, the user may have to manually input the goal into the system. In yet another example, the system may extract the user's goals from social media, the user's emails, goals inputted into another program, a database stored by the user's trainer, another source, or combinations thereof.

The physiological parameter and the portions of the workout history may be identified from the user's profile through any appropriate mechanism. In one example, the system sends crawlers into the user's personal profile which attaches metadata to pieces of information that are desirable for determining the user's supplement formula. For example, the crawlers may tag the user's weight, the user's heart rate, the types of exercise performed by the user, and so forth. In one example, the type of workout may be tagged. Each tag within the recent time period may be counted to determine the user's workout frequency. Also, different tags may be associated with aerobic workouts from anaerobic workouts. Counting the tags associated with aerobic workouts verses the anaerobic workouts can assist the system in determining the types of workouts that are commonly performed by the user. Each of the tagged workouts may also include metadata associated with the user's physiological parameters. For example, a workout that resulted in the user having a highly elevated heart rate can be designated in the metadata as an intense workout whereas a workout that resulted in the user having a low heart rate can be designated in the metadata as an easy workout. Additionally, the workout duration may also be tagged in the personal profile. The workout duration combined with the user's workout temperature may be used to determine the volume of fluids, salts, and other nutrients that were likely lost during the user's workout.

In addition to tagging information related to the user's workouts, other types of activities performed by the user may also be tagged. In one example, parameters dealing with the user's sleep may be tagged. The metadata associated with the user's sleep may include information about the user's sleep temperature, the duration of the user's sleep, the restfulness of the user's sleep, and other types of sleep parameters.

In one example, each of the metadata includes values that are associated with a specific type of nutrient. For example, each type of workout may receive a value attributed to a sodium depletion. For an aerobic workout, a sodium value may be incremented by one for every five minutes that a user's heart rate is over a predetermined threshold. Thus, if the user's heart rate is over the predetermined threshold for an hour, the sodium value may be 12. Continuing with this example, the sodium value may be added to the other sodium values from other workouts within the time period. In this example, a total sodium value may be determined for the entire period. The sodium value may correspond to a baseline sodium volume that is to be included in the nutritional supplemental formula. The baseline may be adjusted depending on the user's health history, fatigue experienced during the workouts, requests by the user, requests by the user's trainer, the user's goals, other factors, or combinations thereof. In this example, each of the types of nutrients to be included in the nutritional supplement combination may each have its own value per workout that is additive over the time period as explained in the above example focused on sodium.

In other examples, the workouts are assigned a value per nutrient. The values are averaged and the average value corresponds to that nutrient's baseline value for generating the formula.

The nutrient values may be adjusted based on certain physiological values of the user. For example, the nutrient value may be multiplied by the user's weight and/or the user's lean body mass to determine an adjusted value. In some examples, the adjusted value corresponds to the baseline nutrient value.

In yet another example, the number of anaerobic workouts and the number aerobic workouts are totaled. These totals are adjusted by the user's weight, height, age, body composition, gender, another type of physiological parameter, or combinations thereof. A blend of nutrients may correspond to the total values. In this example, a ratio of anaerobic workouts to aerobic workouts may be used to adjust the formula. For example, a user that performs just anaerobic workouts may cause a specific formula to be generated while a user that performs just aerobic workouts may cause another specific formula to be generated. For those users that have a mixture of anaerobic to aerobic workouts, the system may cause a blend between the two specific formulas to be generated.

In some situations, after the amount of nutrients that the user consumes during his or her workouts is determined, the user's goals are used to adjust the formula. For example, if a user has a goal to increase his lean body mass, additional nutrients may be added that promote muscle growth.

The recent time period may be a time period that corresponds with the amount of supplements that are sent to the user. For example, if the system is set to send a two week supply of supplements to the user, the recent time period may be a two week period. In this example, when the two week supply is projected to run low, the system may access the user's profile for analysis to create the customized supplement formula.

In response to generating the formula, the system can cause the nutritional supplement combination to be created based on the formula. This may occur by opening containers of the nutrients and directing the appropriate nutrient amounts to packaging to be shipped to the user.

The customization system may include a combination of hardware and programmed instructions for executing the functions of the customization system. The customization system may include processing resources that are in communication with memory resources. Processing resources include at least one processor and other resources used to process the programmed instructions. As described herein, the memory resources may represent generally any memory capable of storing data such as programmed instructions or data structures used by the actuation system.

The processing resources may include I/O resources that are capable of being in communication with a remote device that stores the user information, workout history, external resources, databases, or combinations thereof. The remote device may be a mobile device, a cloud based device, a computing device, another type of device, or combinations thereof. In some examples, the customization system communicates with the remote device through a mobile device which relays communications between the customization system and the remote device. In other examples, the mobile device has access to information about the user. The remote device may collect information about the user throughout the day, such as tracking calories, exercise, activity level, sleep, other types of information, or combination thereof.

The remote device may execute a program that can provide useful information to the actuation system. An example of a program that may be compatible with the principles described herein includes the iFit program which is available through www.ifit.com identified above. An example of a program that may be compatible with the principles described in this disclosure is described in U.S. Pat. No. 7,980,996 issued to Paul Hickman.

The processing resources, memory resources, and remote devices may communicate over any appropriate network and/or protocol through the input/output resources. In some examples, the input/output resources includes a transmitter, a receiver, a transceiver, or another communication device for wired and/or wireless communications. For example, these devices may be capable of communicating using the ZigBee protocol, Z-Wave protocol, BlueTooth protocol, Wi-Fi protocol, Global System for Mobile Communications (GSM) standard, another standard, or combinations thereof. In other examples, the user can directly input some information into the actuation system through a digital input/output mechanism, a mechanical input/output mechanism, another type of mechanism, or combinations thereof.

The memory resources may include a computer readable storage medium that contains computer readable program code to cause tasks to be executed by the processing resources. The computer readable storage medium may be a tangible and/or non-transitory storage medium. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory, or combinations thereof.

The profile accessor, may use any appropriate mechanism to access the user's profile. In some examples, the profile accessor includes keys and encryption codes that allow the system to gain access to the personal profile. The physiological parameter identifier may tag pieces of information in the profile that contain physiological information about the user during a workout or the user during a state of rest. The workout history identifier may tag events in the workout history of the user's profile. The consumed nutrient determiner may be used to determine the amount of nutrients that the user has consumed during the recent time period based on the user's physiological parameters and the user workout history.

A fitness goal determiner may gather information from the user's profile to determine the user's fitness goals. In other examples, the user's goals may be obtained through other sources. The user's goals may deal with changing the user's body composition, gaining muscle, losing fat, lowering his or her resting heart rate, increasing his or her VO₂ max, changing the weight amount that the user can lift, reducing the time it takes the user to run or bike a specified distance, sport specific goals, other types of goals, or combinations thereof. The goal nutrient determiner may determine the additional amount of nutrients that are to be added to the formula based on the user's goals. For example, if the user wants to lose fat, the goal nutrient determiner may adjust the amount of nutrients to promote fat lose.

In some situations, the profile accessor causes the tagged information or requested information to be sent to the system for analysis. In other examples, this information is passively sent to the system without request from the system. The user may manually request that the system prepare supplements for shipping. In this example, the user's request triggers the profile accessor to start the process of gathering information.

In some scenarios, the user inputs the amounts and types of food that he or she eats into the system. In this situation, the system may determine the types of nutrition that the user needs using the user's diet as a factor. For example, if the system determines that the user has not consumed an appropriate amount of a certain type of food to provide a desired nutrient, the system can cause the lacking nutrient to be added with the other nutrients. Additionally, the user's dietary input may be tracked by any number of systems, and any dietary nutritional deficiencies can be accounted for by the formula generator. For example, if a user's diet is tracked and the system identifies a lack of calcium in a user's diet, calcium may be added to the customized nutritional supplement to provide the user with more complete nutrition.

A formula generator represents programmed instructions that cause the processor, when executed, to generate a nutritional supplement formula based on the analyzed information from the user's profile and, in some cases, the user's fitness goals from other sources. A nutrient director causes the nutrients to be directed for packaging, and a shipping preparer prepares the nutrients for shipping. In some cases, the shipping prepare prints a label with the user's mailing address or otherwise causes the user's address to be affixed to the packaging.

An orifice controller may represent programmed instructions that, when executed, cause the processing resources to control the orifice of each container to ensure that the appropriate amount of nutrient is dispensed. For example, the orifice controller may be in communication with an actuator that can cause the orifice to open for a selected amount of time to cause an appropriate amount of nutrient to be dispensed before the orifice closes. In some cases, the orifice controller is in communication with a nutrient sensor that determines the overall volume of nutrient in the container to ensure that the appropriate amount of nutrient has been dispensed. In some examples, the actuators are also used to position the containers over a platform that supports the cup. In this manner, the cartridges can be moved as appropriate to be out of the way when not dispensing the nutrient.

Further, the memory resources may be part of an installation package. In response to installing the installation package, the programmed instructions of the memory resources may be downloaded from the installation package's source, such as a portable medium, a server, a remote network location, another location, or combinations thereof. Portable memory media that are compatible with the principles described herein include DVDs, CDs, flash memory, portable disks, magnetic disks, optical disks, other forms of portable memory, or combinations thereof. In other examples, the program instructions are already installed. Here, the memory resources can include integrated memory such as a hard drive, a solid state hard drive, or the like.

In some examples, the processing resources and the memory resources are located within the treadmill, a mobile device, an external device, another type of device, or combinations thereof. The memory resources may be part of any of these device's main memory, caches, registers, non-volatile memory, or elsewhere in their memory hierarchy. Alternatively, the memory resources may be in communication with the processing resources over a network. Further, data structures, such as libraries or databases containing user and/or workout information, may be accessed from a remote location over a network connection while the programmed instructions are located locally.

In one example, a method for customizing a nutritional supplement to a user includes identifying at least one physiological parameter experienced by the user within a recent time period from a web based program, identifying at least a portion of a workout history from the recent time period from the web based program, identifying a fitness goal of the user from the web based program, combining nutritional supplements to form a nutritional supplement combination based on the at least one physiological parameter, the portion of the workout history, and the fitness goal, and shipping the nutritional supplement combination to the user.

Any appropriate physiological parameter experienced by the user may be identified during a workout or another time. A non-exhaustive list of physiological parameters may include an amount of sleep of the user within the recent time period, a heart rate of the user during an event in the workout history, a user temperature during sleep within the recent time period, a user temperature during an event in the workout history, a user respiration rate during an event in the workout history, a user weight within the recent time period, a user gender, a user body composition within the recent time period, another physiological parameter, or combinations thereof.

The nutrients in the nutritional supplement combination may include any appropriate type of nutrients. For example, the nutrients may include creatine kinase, protein, water, carbohydrates, sodium, chloride, potassium, magnesium, calcium, phosphate, vitamin A, vitamin, B, vitamin C, vitamin D, vitamin E, vitamin K, thiamin, riboflavin, niacin, folate, pyridoxine, cobalamin, pantothenic acid, biotin, iron, zinc, copper, selenium, iodine, fluoride, chromium, manganese, molybdenum, magnesium, glutamine, glycine, carnitine, arginine, taurine, lysine, lutein, calcium, other vitamins, other minerals, other amino acids, other proteins, other herbs, other hormones, other molecules, other carbohydrates, other nutrients, or combinations thereof.

What is claimed is:

1. A system for customizing nutritional supplement, comprising:
a processor and memory, the memory includes programmed instructions that, when executed, cause the processor to:
access a profile of a user in a web based program;
identify, from the profile, at least one physiological parameter experienced by the user within a recent time period;
identify, from the profile, at least a portion of a workout history from the recent time period;
identify a fitness goal of the user; and
generate a customized nutritional supplement formula based on the at least one physiological parameter, the portion of the workout history, and the fitness goal.

2. The system of claim 1, wherein the programmed instructions cause the processor, when executed, to:
determine consumed nutrient amounts by the user based on the at least one physiological parameter and the workout history;
determine goal nutrient amounts to achieve the fitness goal; and
combine the consumed nutrient amounts and the goal nutrient amounts.

3. The system of claim 1, wherein identifying the fitness goal includes extracting goal information from the profile.

4. The system of claim 1, wherein identifying the fitness goal includes receiving the fitness goal from a source other than the profile.

5. The system of claim 1, wherein identifying the at least one physiological parameter includes identifying an amount of sleep of the user within the recent time period.

6. The system of claim 1, wherein identifying the at least one physiological parameter includes identifying a heart rate of the user during an event in the workout history.

7. The system of claim 1, wherein identifying the at least one physiological parameter includes identifying a user temperature during sleep within the recent time period or includes identifying a user temperature during an event in the workout history.

8. The system of claim 1, further comprising:
instructions that, when executed, cause the processor to:
track a dietary input of the user;
identify any dietary nutritional deficiencies of the user; and
add a nutrient to the customized nutritional supplement formula in response to the identified dietary nutritional deficiency.

9. The system of claim 1, wherein identifying the at least one physiological parameter includes identifying a user respiration rate during an event in the workout history.

10. The system of claim 1, wherein identifying the at least one physiological parameter includes identifying a user weight within the recent time period.

11. The system of claim 1, wherein identifying the at least one physiological parameter includes identifying a user gender.

12. The system of claim 1, wherein identifying the at least one physiological parameter includes identifying a user body composition within the recent time period.

13. The system of claim 1, further including:
multiple containers, wherein each of the multiple containers is filled with at least one type of nutrient;
a closable orifice in at least one of the multiple containers;
wherein the memory includes programmed instructions that cause the processor, when executed, to direct the at least one type of nutrient into a supplement combination based on the customized nutritional supplement formula.

14. The system of claim 1, wherein the memory includes programmed instructions that, when executed, cause the processor to prepare a supplement combination based on the customized nutritional supplement formula to be shipped to the user.

15. The system of claim 14, wherein the preparing the supplement combination includes printing a shipping address associated with the user.

16. A system for customizing nutritional supplement, comprising:
a processor and memory, wherein the memory includes programmed instructions that, when executed, cause the processor to:
access a profile of a user in a web based program;
identify, from the profile, at least one physiological parameter experienced by the user within a recent time period;
identify, from the profile, at least a portion of a workout history from the recent time period;
determine consumed nutrient amounts by the user based on the at least one physiological parameter and the workout history;
identify a fitness goal of the user;
determine goal nutrient amounts to achieve the fitness goal; and
combine the consumed nutrient amounts and the goal nutrient amounts to generate a customized nutritional supplement formula.

17. The system of claim 16, further including:
multiple containers, wherein each of the multiple containers is filled with at least one type of nutrient;
a closable orifice in at least one of the multiple containers;
wherein the memory includes programmed instructions that cause the processor, when executed, to direct the at least one type of nutrient into a supplement combination based on the customized nutritional supplement formula.

18. The system of claim 16, wherein the memory includes programmed instructions that, when executed, cause the processor to prepare a supplement combination based on the customized nutritional supplement formula to be shipped to the user.

19. The system of claim 18, wherein the preparing the supplement combination includes printing a shipping address associated with the user.

20. A system for customizing nutritional supplement, comprising:
multiple containers, wherein each of the multiple containers is filled with at least one type of nutrient;
a closable orifice in at least one of the multiple containers;
a processor and memory, the memory includes programmed instructions that, when executed, cause the processor to:
access a profile of a user in a web based program;
identify, from the profile, at least one physiological parameter experienced by the user within a recent time period;
identify, from the profile, at least a portion of a workout history from the recent time period;
determine consumed nutrient amounts by the user based on the at least one physiological parameter and the workout history;
identify a fitness goal of the user;
determine goal nutrient amounts to achieve the fitness goal;
combine the consumed nutrient amounts and the goal nutrient amounts to generate a customized nutritional supplement formula;
direct the at least one type of nutrient into a supplement combination based on the customized nutritional supplement formula; and
prepare a supplement combination to be shipped to the user by printing a shipping address associated with the user.

21. A method for customizing a nutritional supplement to a user, comprising:
identifying at least one physiological parameter experienced by the user within a recent time period from a web based program;
identifying at least a portion of a workout history from the recent time period from the web based program;
identifying a fitness goal of the user from the web based program;
combining nutritional supplements to form a nutritional supplement combination based on the at least one physiological parameter, the portion of the workout history, and the fitness goal; and
shipping the nutritional supplement combination to the user.
